(19) United States
(12) Patent Application Publication
(10) Pub. No.: US 2011/0217259 A1
(43) Pub. Date: Sep. 8, 2011

(54) IL-16 as a target for diagnosis and therapy of hematological malignancies and solid tumors

(76) Inventors: Djordje ATANACKOVIC, Hamburg (DE); Nicolaus Kroger, Hamburg (DE)

(21) Appl. No.: 12/716,481

(22) Filed: Mar. 3, 2010

(51) Int. Cl. A61K 38/00; A61K 38/20; A61K 39/395; A61K 35/28; C12Q 1/68; C12Q 1/02; A61K 31/7088; A61P 7/00

(52) U.S. Cl. 424/85.2; 424/158.1; 424/178.1; 424/93.7; 435/29; 514/44 R; 514/44 A; 514/19.3; 514/19.4; 514/19.5; 435/6.14; 435/6.17

(57) ABSTRACT

The present invention relates to the field of diagnosis and therapy of hematological malignancies, such as multiple myeloma, as well as solid tumors based on cytokine interleukin-16 (IL-16) and agents specifically targeting this antigen or cells expressing the same, e.g., antibodies. The inventors were able to prove that IL-16 is expressed and secreted at high levels by myeloma cells. Most importantly, the inventors have demonstrated for the first time that IL-16 supports the proliferation of the malignant cells. Therefore, this cytokine represents a particularly advantageous target in cancer therapy and diagnosis.

### FIELD OF THE INVENTION

The present invention relates to the field of diagnosis and therapy of hematological malignancies, such as multiple myeloma, as well as solid tumors, based on the cytokine interleukin-16 (IL-16) and agents specifically targeting this antigen or cells expressing the same, e.g., antibodies. The inventors were able to prove that IL-16 is expressed and secreted at high levels by myeloma cells. Most importantly, the inventors have demonstrated for the first time that IL-16 supports the proliferation of the malignant cells. Therefore, this cytokine represents a particularly advantageous target in cancer therapy and diagnosis.

### BACKGROUND OF THE INVENTION

Hematological malignancies originate from blood cells and bone marrow cells as well as immune cells within lymph nodes. Multiple myeloma is one example of a hematological malignancy, other diseases in this group comprise acute myeloid leukemia, chronic myeloid leukemia, acute lymphatic leukemia, chronic lymphatic leukemia, Hodgkin's disease and Non-Hodgkin lymphoma and myelodysplastic syndrome. Myeloproliferative diseases are related entities. While there are treatment options for some of these diseases, further therapeutic approaches are urgently needed.

Multiple myeloma (MM) is a clonal plasma cell malignancy with an incidence of approximately 15,000 new cases per year in the U.S. alone. Multiple myeloma is characterized by an accumulation of mature plasma cells in the bone marrow and the detection of a monoclonal protein (paraprotein or M protein) in the serum or urine. Progression of myeloma eventually leads to bone destruction, symptoms of bone marrow failure, hypercalcemia, renal insufficiency, and lytic bone lesions. Several multiple myeloma-related plasmaproliferative disorders, such as mono-clonal gammopathy of undetermined significance (MGUS), smoldering myeloma (SMM), and indolent multiple myeloma (IMM) are characterized by the detection of paraprotein in the serum or urine without the clinical features of MM. MGUS, a clinically benign precursor condition of MM, is more common than MM and occurs in 1% of the population over the age of 50 years and 3% of those over 70 years. The prevention of progression from MGUS, usually occurring at a rate of 1-2% per year, thus would have a significant impact on the morbidity and mortality of myeloma and the older-aged population in general.

Within the last decade, treatment strategies targeting specific biological mechanisms such as angiogenesis have been developed and seem to significantly improve the outcome of MM patients. However, even after application of strategies incorporating new drugs such as bortezomib, thalidomide, or lenalidomide most patients will eventually relapse and succumb to the disease. One reason for this high rate of relapse even after multimodal and/or high dose chemotherapy plus autologous stem cell transplantation (autoSCT) might be the persistence of bone marrow-residing myeloma stem cells which seem to escape standard chemotherapeutic agents. As a consequence, myeloma remains essentially incurable by conventional anti-tumor therapy and patients continue to show a median survival of only 5 years. In conclusion, new therapeutic targets, expressed by the bulk of end-stage myeloma cells as well as their dormant progenitors, are needed for the development of treatments capable of eradicating minimal residual disease leading eventually to a an increased rate of cures or at least prolonged remission.

Targeting myeloma-related surface molecules or growth factors by antibodies or immunoconjugates represents an attractive therapeutic modality for the eradication of MM. Accordingly, therapeutic antibodies against a variety of myeloma-associated antigens such as IL-6, CD40, CD56, CD138, insulin-like growth factor type I, and CD20 have been developed, however, none of these therapeutics have, so far been proven to be clinically effective and, accordingly, alternative myeloma-related proteins are urgently needed as potential targets.

To address these severe problems in the current treatment of multiple myeloma and other malignancies, the development of alternative and more targeted approaches that can be safely applied in such settings is essential. Furthermore, it would be beneficial to develop a means of diagnosis that only requires a blood sample from the respective patients.

In light of the state of the art, the inventors developed a novel and advantageous target for therapy and diagnosis of hematological malignancies, such as multiple myeloma, as well as of solid tumors.

### SUMMARY OF THE INVENTION

The inventors were able to prove that IL-16 is expressed in and secreted by tumor cells, in particular malignant cells derived from patients with multiple myeloma. While preliminary indications that IL-16 is expressed by some tumors existed (Alonsi et al., 2007; Bellomo et al., 2007; Alexandrakis et al., 2004; Koike et al., 2002; Magrangeas et al., 2003, Cao et al., 2009), the inventors have shown for the first time that IL-16 plays an important role in promoting the growth of the tumor cells and that anti-IL-16 approaches, such as transfection with inhibiting RNA or treatment with antibodies directed against IL- and/or its receptors, have a growth-inhibiting effect on myeloma cells. Based on these findings of the inventors, it has for the first time become clear that IL-16 represents a very promising target for the therapy of cancers, in particular hematological malignancies such as multiple myeloma.

In one aspect, the present invention provides a method of diagnosing a malignancy, e.g., a hematopoetical malignancy or a solid tumor, in a subject, comprising steps wherein the expression of IL-16 is detected in a sample obtained from the subject. The subject may be a patient potentially suffering from the respective malignancy, or a patient with a pre-malignant condition, such as MGUS. The IL-16 may be human IL-16, e.g., as disclosed in Baier et al., 1997.

The sample may be a blood sample (e.g., serum, plasma, peripheral blood mononuclear cells (PBMC), whole blood including mononuclear cells), a bone marrow sample (comprising, e.g., serum, plasma, mononuclear cells), a sample of lymphoid tissue (e.g., lymph nodes or spleen), or a tissue sample from an other organ infiltrated by the malignancy, or it may be derived from such a sample. The sample may be contacted with an agent capable of binding to IL-16 protein, e.g., an antibody, or a nucleic acid capable of hybridizing to IL-16 RNA or cDNA. The expression may be detected on the protein level, e.g., by enzyme-linked immunosorbent assay (ELISA), antibody array, immunohistochemistry, flow cytometric analysis, cytospin or an immunoblot, such as a Western blot.

The expression may also be detected on the RNA level, e.g., by amplification and/or hybridization techniques, e.g., Northern Blot, array technology and/or RT-PCR. Real time-PCR may be employed, e.g., a light cycler™ (Roche). Examples of suitable primers and conditions for detecting expression are disclosed in Cho et al. (2008), but other suitable primers or probes capable of hybridizing to the IL-16 nucleic acid under suitable conditions can be prepared by one of skill in the art.

Determining the expression of IL-16 in such a sample may be useful for diagnosing a hematological malignancy or a solid tumor in a subject. A high level of IL-16 expression indicates a high risk that the subject has a hematological malignancy or a solid tumor.

Determining the expression of IL-16 in such a sample may also be useful for determining the likelihood of progression from a premalignancy (e.g., MGUS) to a malignancy (i.e., a clinically relevant cancer, e.g., MM) in a subject, comprising the collection of a sample from said subject and determining expression of IL-16 in said sample. The likelihood of progression is high if there is a high expression of IL-16 in the cells (e.g. bone marrow cells) or in other types of patient material (bone marrow plasma, plasma or serum derived from peripheral blood, urine), e.g., a significantly higher expression than the average expression in samples from healthy subjects and/or in earlier samples from the same patient.

Determining the expression of IL-16 in such a sample may also be useful for determining progression or regression of a hematological malignancy or a solid tumor in a subject, wherein expression of IL-16 is determined in a minimum of two samples obtained from the subject at different time points, wherein a higher amount of IL-16 in the later sample/samples compared to the earlier sample indicates progression or relapse and a lower amount of IL-16 in the later sample when compared to the earlier sample indicates regression. This method can be useful for determining, e.g., success of an anticancer treatment. Samples can, e.g., be taken before, during and/or after treatment.

In another aspect, the invention provides a method of treating multiple myeloma, another hematological malignancy, or a solid tumor, comprising administering to a subject an effective amount of an agent capable of specifically targeting IL-16 or of targeting cells expressing IL-16. Also provided is a method of preventing progression from a premalignancy to a malignancy, comprising administering to a subject an effective amount of an agent capable of specifically targeting IL-16 or cells expressing IL-16.

The method can involve the administration of an IL-16 inhibitor to an individual diagnosed with the respective hematological malignancy, such as MM, or a solid tumor. An agent capable of specifically targeting IL-16 or cells expressing this cytokine, in particular an agent capable of specifically binding to IL-16, may be administered to a the respective patient.

The invention provides a pharmaceutical composition for the treatment of a solid tumor or a hematological malignancy, e.g., multiple myeloma, or for the diagnosis of a hematological malignancy, wherein the composition comprises an agent capable of specifically targeting IL-16 or cells expressing IL-16, such as agent preventing binding of IL-16 to one or more IL-16 receptors, e.g., an anti-IL-16 antibody.

In another aspect, the current invention provides a method or pharmaceutical composition for employing an IL-16-targeted approach for preventing progression from a premalignancy, such as MGUS, to a clinically relevant cancer/malignancy, such as MM among hematological malignancies.

Interleukin-16 is the cytokine the inventors found to be expressed in and secreted by hematological malignancies such as multiple myeloma. It preferably comprises human IL-16 (Baier et al., 1997) or consists thereof.

The agent may be a small molecule specifically binding to IL-16, an anti-IL-16 antibody or a natural or soluble receptor of IL-16 (e.g., comprising an IL-16 binding portion of CD4 or CD9), or an antibody/antibodies directed against one or two of the IL-16 receptors (CD4 and/or CD9), preferably, an antagonistic antibody directed against the receptor(s). The agent targeting IL-16 may be an anti-IL-16 receptor antagonist or an IL-16 TRAP. The agent may comprise an antibody, preferably, a monoclonal and/or recombinant antibody. In particular, for therapeutic approaches, the antibody may be a chimeric, human or humanized antibody, which prevents or reduces immunogenicity. Preferably, the antibody is a recombinant monoclonal human or humanized antibody. The antibody may also be a single chain antibody, an antigen binding fragment of an antibody, e.g., a Fab of F(ab)2 fragment or a polyclonal antibody.

The invention provides an antibody capable of specifically binding to IL-16, in particular to the C-terminal portion thereof, as described below. An anti-IL-16 antibody may also bind to the other, more N-terminal regions of IL-16.

The invention provides an antagonistic anti-CD-4 antibody capable of preventing binding of IL-16 to the receptor for use in treating a malignancy, in particular a hematological malignancy such as multiple myeloma, or for preventing progression of a premalignancy to a malignancy. The invention also provides an antagonistic anti-CD-9 antibody capable of preventing binding of IL-16 to the receptor for use in treating a hematological malignancy such as multiple myeloma, or for preventing progression of a premalignancy to a malignancy.

The agent, e.g., an agent specifically binding to IL-16 (such as an anti-IL-16 antibody or a soluble IL-16 receptor), may be coupled to an active compound, e.g., a cytotoxic compound, an enzyme, such as an enzyme that converts a non-toxic compound to a cytotoxic compound, a radioisotope or a detectable, e.g., fluorescent, label. Examples of active compounds suitable for therapeutic purposes are known from the state of the art, e.g., calicheamin, esperamin, methotrexate, doxorubicin, daunorubicin, melphalan, vincristin, cyclophosphamide, chlorambucil, cytarabin, vindesine, mitomycin C, cisplatin, etopside, bleomycin and fluorouracil. Radioisotopes include: ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³_{I,} ⁷⁷Br, ¹⁵³_{Sm,} ¹⁶⁶Bo, ⁶⁴Cu, ²¹²Pb, ²²⁴Ra and ²²³Ra.

The agent targeting IL-16 may alternatively inhibit IL-16 expression on the RNA and/or protein level. For example, the agent may comprise inhibitory RNA or antisense RNA. Use of inhibitory RNA against cytokines is known in the state of the art (e.g., WO 2006060598). Exemplary methods for inhibiting expression of IL-16 on the RNA level are disclosed below, but alternatives will be evident to the skilled person.

The agent may target cells expressing IL-16. The agent may comprise IL-16-specific T cells, which can be autologous or allogeneic IL-16-specific T cells. The IL-16-specific T cells may be generated in vivo, e.g., by vaccination of the patient or a different human individual sharing at least one, preferably, all HLA types. Such T cells can be isolated and further stimulated/propagated in vitro. IL-16-specific T cells may also be generated ex vivo, e.g., by transduction of T cells with an IL-16-specific T cell receptor [9] and/or in vitro induction and/or expansion of IL-16-specific T cells, which may, e.g., be isolated from the patient. IL-16 protein (or peptide) or a nucleic acid (RNA or DNA) encoding the same, as defined below, may be used for immunization and/or in vitro stimulation, e.g., a protein comprising a fragment of IL-16 comprising at least one T cell epitope thereof, or a protein having at least 90% amino acid identity to the sequence of human IL-16 as disclosed, e.g., in Baier et al. (1997).

The agent is to be administered in an effective amount, i.e. administration of the agent should lead to depletion of IL-16-positive cells from the patient. In one embodiment of the invention, the pharmaceutical composition is formulated for reduction and/or depletion of IL-16-expressing cells from the patient in vivo. The pharmaceutical composition may be formulated for infusion. Methods of administration, treatment regimens and dosing may be selected by the skilled person as known for similar agents from the state of the art. The pharmaceutical formulation may also be used in combination with other treatment regimens and medicaments. The pharmaceutical composition may be employed in induction therapy and/or consolidation therapy and/or maintenance therapy.

In another embodiment, the invention provides a method of treating a hematological malignancy or of preventing progression from a premalignancy to a malignancy, comprising depleting cells expressing IL-16 and/or depleting soluble IL-16 protein ex vivo. The invention also provides a pharmaceutical composition formulated for reduction and/or depletion of IL-16-positive cells or IL-16 protein from the patient's body, i.e. the peripheral blood, ex vivo. For example, the agent capable of binding to IL-16, such as an antibody, may be linked to a solid carrier and/or magnetic beads under conditions suitable for binding to IL-16 present on and/or secreted by the patient's cells. Patient-derived material, i.e. peripheral blood, may be brought into contact with the carrier, to be re-infused into the patient after separation from the carrier and depletion of the IL-16 expressing cells and/or soluble IL-16 protein. Of course, other options known in the state of the art can be adapted for depletion of IL-16 and interleukin-16-expressing cells.

The invention further provides use of a composition comprising IL-16 protein and/or a nucleic acid encoding IL-16 protein for the preparation of a prophylactic or therapeutic vaccine for treatment of a hematological malignancy or for in vitro stimulation of allogeneic or autologous T-cells. The invention provides a method of treating a hematological malignancy or a solid tumor or for preventing progression from a premalignancy to a malignancy, comprising administering to a subject an effective amount of a composition comprising an IL-16 protein and/or a nucleic acid encoding an IL-16 protein.

In the context of the invention, the IL-16 protein comprises a sequence having at least 80%, at least 90%, least 95% or at least 98% or at least 99% amino acid identity to a protein of having the sequence of human IL-16 (Baier et al., 1997), or a fragment thereof comprising at least one T-cell epitope and/or at least one B-cell epitope of IL-16. The IL-16 protein may comprise the sequence of a IL-16 precursor protein or of a mature human IL-16 protein, a natural allele thereof, or a sequence of at least one T-cell epitope and/or B-cell epitope. Most preferably, IL-16 protein comprises the sequence of mature human IL-16, with the consensus sequence in CCDS10317.1 (or, e.g., Baier et al., 1997). IL-16 may be differentially processed, e.g. to transcript variant 1 and 2. In the context of the invention, IL-16 refers to all isoforms, in particular the mature soluble isoforms. In one embodiment, IL-16 comprises the C-terminal part of IL-16, in particular, of the sequence in CCDS10317.1, preferably comprising the 121 C-terminal amino acids. It is believed that the C-terminal part of IL-16 plays an important role in the function of IL-16 as a chemoattractant for i.e. immune cells, while the N-terminal part may play a role in cell cycle control. In one embodiment, the IL-16 protein comprises the sequence of one or more T cell epitope and/or B cell epitope. It is known that one or more amino acid exchanges, deletions or introductions often do not change the structure, immunological properties and/or binding characteristics of a protein, in particular with conservative amino acid substitutions. The IL-16 protein, in particular the sequence having at least 90% amino acid identity to human IL-16 protein, is preferably capable of being specifically recognized by an antibody against IL-16 of human IL-16, e.g., a polyclonal antibody preparation commercially available from Abnova, Taiwan. Preferably, an IL-16 protein comprises more than one T-cell epitope, e.g., one or more CD4 T cell epitopes and/or one or more CD8 T cell epitopes. For the purposes of vaccination, it is preferred to use the complete IL-16 protein (or nucleic acids encoding it) or proteins or peptides comprising a selection of T cell epitopes appropriate for the most common HLA types.

IL-16 proteins are known in the state of the art and disclosed, e.g., in DE 196 49233 or DE 19617203.

In the context of the invention, IL-16 proteins include fusion proteins of IL-16 proteins or fragments, e.g., fragments thereof comprising a B-cell epitope and/or T-cell epitope. Examples are fusion proteins with a His-Tag, GST-Tag, FLAG-Tag, GFP-tag or with components intended to enhance the immune response.

The vaccines may comprise at least one IL-16 protein and/or at least one nucleic acid encoding an IL-16 protein, as defined above. Methods suitable for vaccination with nucleic acids, e.g., DNA or RNA are known in the state of the art. For example, viral vectors, such as adenoviral vectors or liposomes can be used for delivery of the effective agent for vaccination. A preferred nucleic acid encodes at least one IL-16 protein as described above. In one embodiment, the nucleic acid comprises the sequence of the wildtype human IL-16 cDNA or DNA, as disclosed in Baier et al. (1997) or a fragment thereof encoding a B cell epitope and/or a T cell epitope. Due to the degeneracy of the genetic code, different codons may be used to encode human IL-16 protein, accommodations may be made for introduction of a higher GC content, which is known to enhance immunogenicity. Often, more than one, e.g., two of three doses of a vaccine of the invention are administered to boost immunogenicity.

The cancer targeted by the invention can be a solid tumor or a hematological malignancy. In the context of the invention, the hematological malignancy may be multiple myeloma (MM), indolent myeloma, smoldering myeloma, acute myeloid leukemia (AML), chronic myeloid leukemia (CML), acute lymphatic leukemia (ALL), chronic lymphatic leukemia (CLL), cutaneous T-cell leukemia (e.g., Sezary syndrome), hairy cell leukemia, Hodgkin's disease, Non-Hodgkin lymphoma, myelodysplastic syndrome (MDS) or myelo-proliferative disease. In one preferred embodiment, the hematological malignancy is multiple myeloma.

One example of a premalignancy is monoclonal gammopathy of undetermined significance (MGUS), which may progress to multiple myeloma.

In the context of the invention, the solid tumor may be colorectal carcinoma, breast carcinoma, lung carcinoma (small-cell lung cancer and non-small cell lung cancer), head and neck cancer, glioblastoma multiforme, sarcoma, cervical cancer, endometrial cancer, cancer of the pancreas, thyroid, stomach, bladder, skin, breast, prostate, ovary, kidney, or liver.

In the context of the invention, the subject preferably is a human subject, but the subject may also be, e.g., a mouse, a rat, a pig, a cat, a dog, a horse, cattle or an ape.

### BRIEF DESCRIPTION OF THE FIGURES

FIG. 1 shows IL-16 is constitutively produced by myeloma cell lines. As shown in FIG. 1A using an antibody array, high concentrations of cytokine IL-16 in the culture supernatant of myeloma cell line EJM were observed. A section of the whole array containing 10 duplicate spots is shown. IL-16-specific spots are indicated by a square. As shown in FIG. 1B, expression of IL-16 was examined in 10 myeloma cell lines applying conventional RT-PCR (upper row) and western blot (lower rows). Unstimulated CD8+ T cells were used as positive controls and housekeeping gene beta actin (ACTB) served as an internal marker for quality of the total protein. As shown in FIG. 1C, cytoplasmatic staining followed by flow cytometry confirmed on a per-cell level that all myeloma cell lines strongly expressed IL-16 protein.

FIG. 2 shows expression of IL-16 among healthy tissues is restricted to lymphatic tissues. Expression of IL-16 RNA was evaluated in a wide variety of human tissues applying quantitative PCR and results are shown as copies of the target gene in relation to copies of housekeeping gene GAPDH. Results indicated that IL-16 was markedly overexpressed in lymphatic tissues.

FIG. 3 shows primary myeloma cells of in the bone marrow of MM patients strongly express IL-16. As shown in FIG. 3A, in order to answer the question whether the IL-16 expressed in the BM of MM patients was primarily produced by the malignant cells, patients were divided into two groups with <30% (N=18) or 30% (N=18) BM-infiltrating plasma cells, respectively. MM patients with higher numbers of BM-infiltrating myeloma cells, in particular, evidenced highly increased levels of IL-16 RNA in comparison to healthy BM donors (N=18), suggesting that the local malignant plasma cells are responsible for the elevated expression of this cytokine. Asterisks indicate significant (**p<0.01) differences between groups. As shown in FIG. 3B, BM-residing plasma cells of 21 patients with established diagnosis of multiple myeloma and one MGUS patient were analyzed by flow cytometry for intracellular expression of IL-16. As the dot plot on the left shows, intensity of IL-16 staining was always higher in myeloma cells, as defined by CD138-positivity, when compared to other cells found within the BM of the myeloma patients. Histograms show results of 8 representative myeloma patients after gating on CD138+ BM plasma cells. Grey areas indicate staining with an irrelevant isotype control, black areas staining with anti-IL-16 antibody.

FIG. 4 shows myeloma cell lines constitutively secrete soluble IL-16. As shown in FIG. 4A, analyses of culture supernatants by ELISA showed that all 11 myeloma lines constitutively secreted soluble IL-16. As shown in FIG. 4B, repeatedly examining culture supernatants of cell lines KMS-12-BM and EJM, IL-16 levels were found to continuously increase until 96 h after culture initiation. As shown in FIG. 4C, 10 different cytokines were separately added to cultures of myeloma cell line EJM and resulting IL-16 concentrations were evaluated 24 hours later. Three cytokines, namely GM-CSF, BAFF and IL-17, further increased production of IL-16 while IFN-α led to a diminished secretion of IL-16 protein. Bars indicate mean (±SEM) IL-16 concentrations and colors of stacked bars indicate concentrations of the respective cytokines added (white: 10 ng/ml; grey: 50 ng/ml; black: 250 ng/ml). The hatched area indicates IL-16 concentration in the supernatant of untreated cells and asterisks mark significant (*p<0.05) differences when compared to untreated cells.

FIG. 5 shows primary myeloma cells within the patients' bone marrow also secrete IL-16. As shown in FIG. 5A, supernatants of 10 myeloma lines were analyzed by western blot for IL-16 protein expression (upper row). In addition, bone marrow plasma samples of 4 myeloma patients and 4 healthy donors (lower row) were analyzed for the presence of IL-16 protein by western blot. In the case of MM patients, BM plasma samples were analyzed undiluted (left band) or diluted at 1:100 (right band). As shown in FIG. 5B, absolute concentrations of IL-16 in the peripheral blood (left box) and in the bone marrow (right box) were compared between myeloma patients (black dots; N=10) and healthy donors (open circles; N=10) in an ELISA assay. Bars show mean values (+SEM) of experiments performed in duplicate and asterisks indicate significant (**p<0.01) differences between groups.

FIG. 6 shows IL-16 expression can effectively be silenced in myeloma cells using siRNA. Myeloma cell lines EJM (upper part) and KMS-12-BM (lower part) were transfected using two siRNA constructs (siRNA-1 and siRNA-2) specific for IL-16 or with scrambled control siRNA. Treatment resulted in knockdown of the expression of IL-16 both cell lines starting 24 h after transfection as indicated by immunoblotting. Knockdown efficiency reached its maximum at 96 h after transfection and lasted at least until day 6.

FIG. 7 shows treatment with inhibitory RNA reduces production and secretion of IL-16 protein by myeloma cells. As shown in FIG. 7A, the effect of IL-16 RNA knockdown on the secretion of soluble IL-16 protein by myeloma cell lines EJM and KMS-12-BM was examined by ELISA. Effects of transfection with unspecific scrambled siRNA are shown as controls. Please note the different scales on the left x-axis (EJM) and on the right axis (KMS-12-BM). As shown in FIG. 7B, supernatants of both myeloma cell lines were examined by western blot in order to find out which forms of soluble IL-16 protein were affected by IL-16 knockdown. As shown in FIG. 7C, a TUNEL assay (right histogram) and staining for Annexin (left histogram) followed by flow cytometry were performed 72 h after transfection of myeloma cell lines KMS-12-BM or EJM with IL-16-specific siRNA. Results of staining with appropriate isotype controls are also indicated.

FIG. 8 shows silencing of IL-16 expression exerts a strong anti-proliferative effect on myeloma cells. As shown in FIG. 8A, the proliferative rate of myeloma cell lines EJM and KMS-12-BM was usually assessed 72 h after transfection with IL-16-specific or control siRNA in an ELISA-based proliferation assay measuring BrdU incorporation. Bars show mean values (+SEM) of three separate experiments and asterisks indicate significant (*p<0.05) differences when compared to untreated cells. As shown in FIG. 8B, the effect of IL-16 knockdown on clonogenic myeloma precursor cells was assessed 72 h after transfection with IL-16-specific or control RNAi in a standard assay measuring clonogenic growth. Colonies consisting of >40 cells were counted at 10 days after culture initiation. Pictures show results of a representative analysis and bars show mean colony numbers (+SEM) of three separate experiments with cell line EJM (left bar) or KMS-12-BM (right bar), resepectively. Asterisks indicate significant (*p<0.05, **p<0.01) differences when compared to untreated cells.

FIG. 9 shows monoclonal antibodies directed against IL-16 and its receptors CD4 and CD9 have a growth-inhibiting effect on myeloma cells. As shown in FIG. 9A, when a monoclonal antibody directed against IL-16 was added to cultures of myeloma cell lines KMS-12-BM, this resulted in a significantly inhibited growth of the malignant cells. Bars show mean values (+SEM) of three separate experiments and asterisks indicate significant (*p<0.05) differences when compared to untreated cells. As shown in FIG. 9B, the application of antibodies against IL-16 receptors CD4 and CD9 had a similar effect. In contrast, the addition of an isotype antibody did not have an impact on the proliferation of myeloma cells. Bars show mean values (+SEM) of three separate experiments and asterisks indicate significant (*p<0.05) differences when compared to untreated cells.

### DETAILED DESCRIPTION OF THE INVENTION

In a comprehensive analysis of multiple myeloma cell lines as well as of primary patient material performed by the inventors, expression and secretion of cytokine IL-16 was detected in myeloma cell lines and their culture supernatants as well as in tumor cells, in the blood and, particularly, in the bone marrow of patients with multiple myeloma. In contrast, IL-16 expression was much lower in the respective tissues from healthy subjects or in other normal organs. Most importantly, the inventors proved for the first time that IL-16 has an important function in malignancies such as multiple myeloma. In particular, they showed that this cytokine supports the proliferation of the malignant cells and that an agent targeting IL-16, such as a monoclonal antibody, is able to suppress tumor growth.

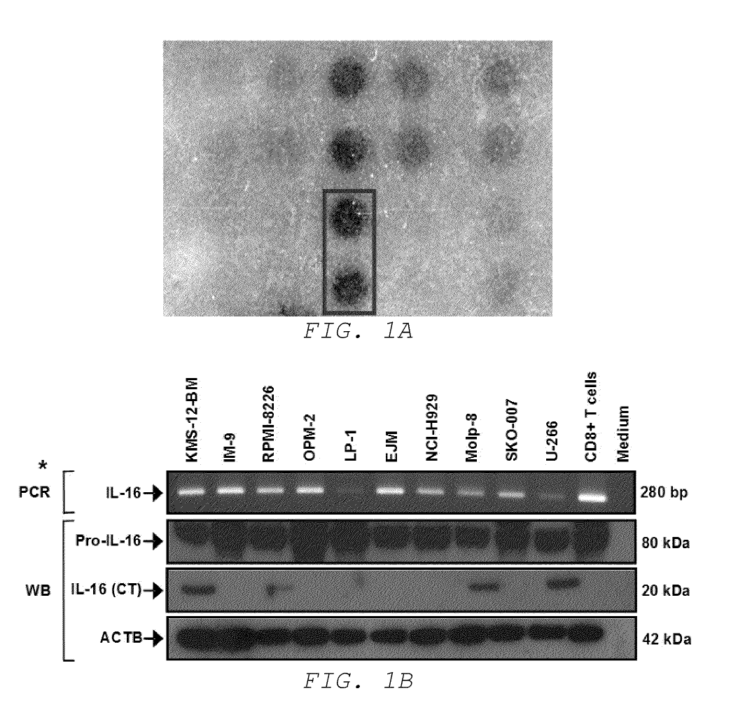

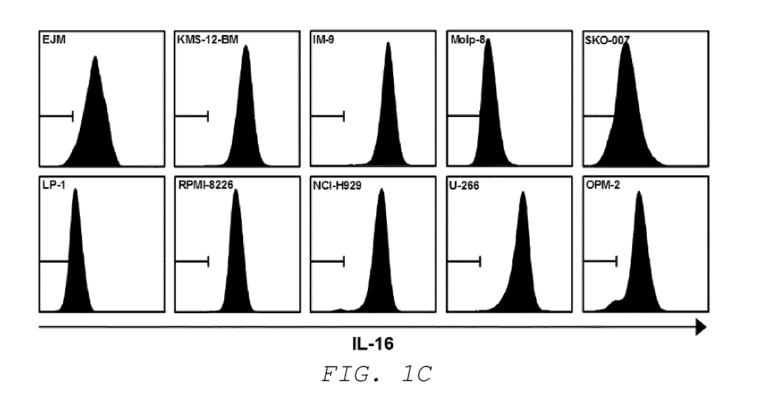

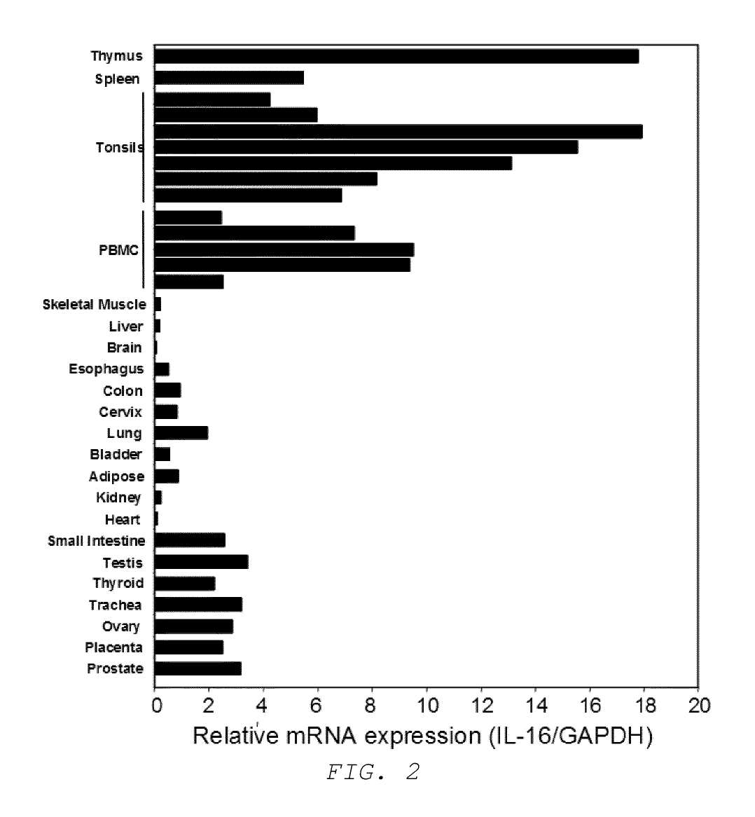

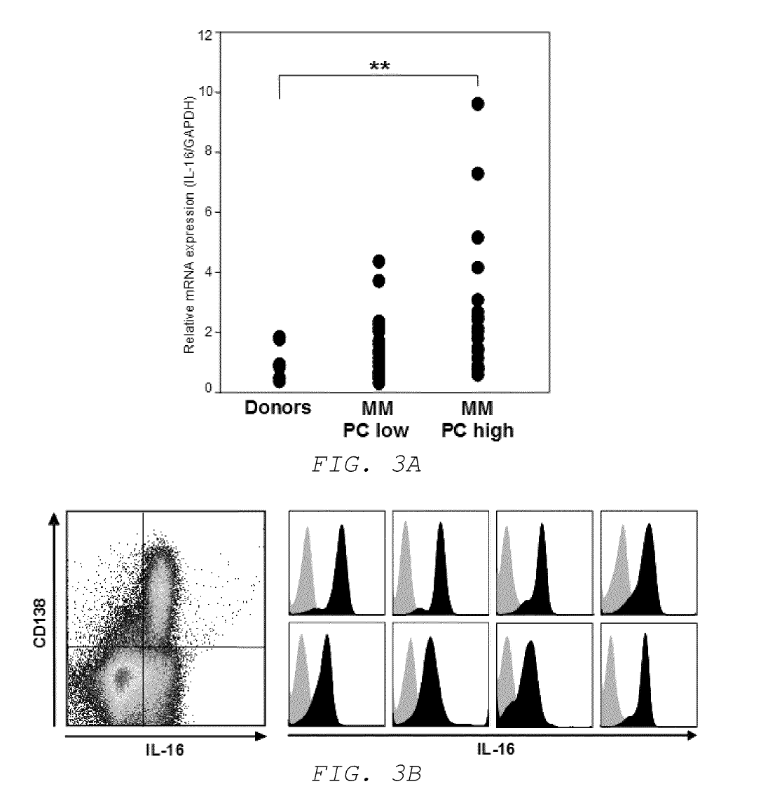

The data provided by the inventors indicate that IL-16 is selectively upregulated in human cancers, in particular in hematological malignancies such as multiple myeloma. The investors have shown that soluble IL-16 protein is expressed and secreted by myeloma cell lines (FIGS. 1A-C). The inventors have confirmed the highly restricted expression pattern of IL-16 in healthy tissues as demonstrated by quantitative RT-PCR (FIG. 2). The investors have demonstrated for the first time that IL-RNA expression is highly increased in the bone marrow of patients with multiple myeloma with bone marrow representing the body compartment where most of the tumor load resides (FIG. 3A). Accordingly, protein expression of IL-16 was demonstrated within malignant cells from myeloma patients (FIG. 3B). Importantly, myeloma cells showed the strongest IL-16 expression of all bone marrow cells allowing for the reliable identification of the tumor cells using flow cytometry (FIG. 3B).

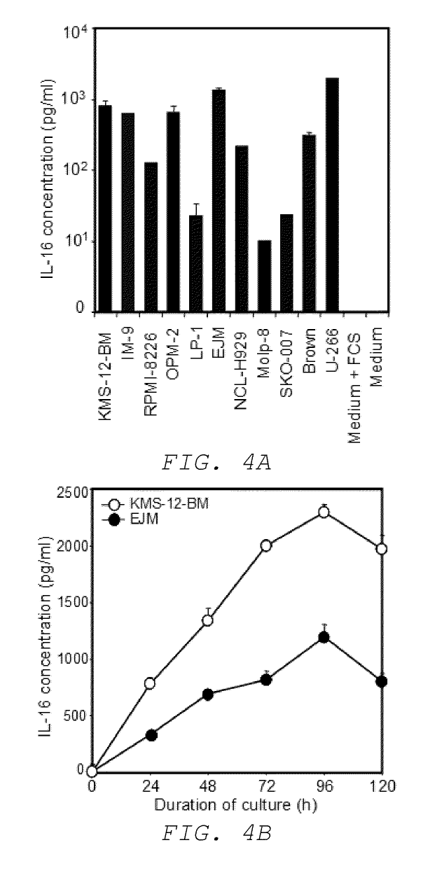

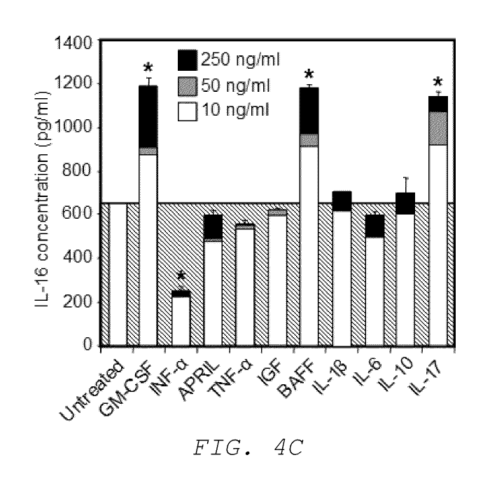

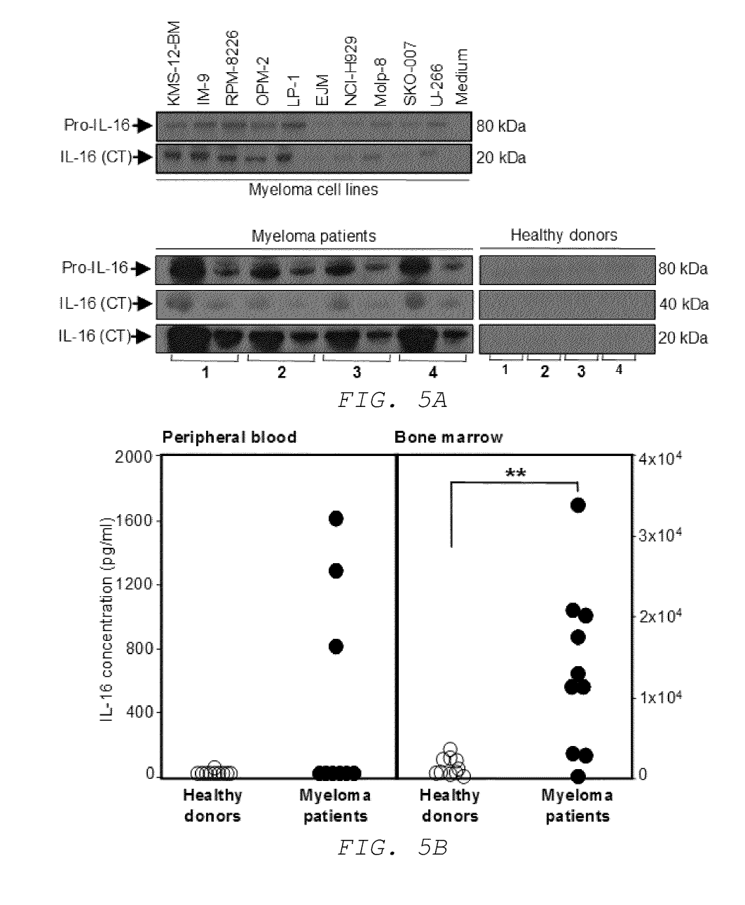

The investors proved for the first time that soluble IL-16 is secreted by myeloma cells (FIGS. 4A+B) and that IL-16 secretion is influenced by other soluble factors such as GM-CSF, BAFF, or IL-17 (FIG. 4C). Secretion of IL-16 by myeloma cells is responsible for increased concentrations of this cytokine in the peripheral blood, and even more pronounced, in the bone marrow of myeloma patients when compared to healthy donors (FIG. 5A+B).

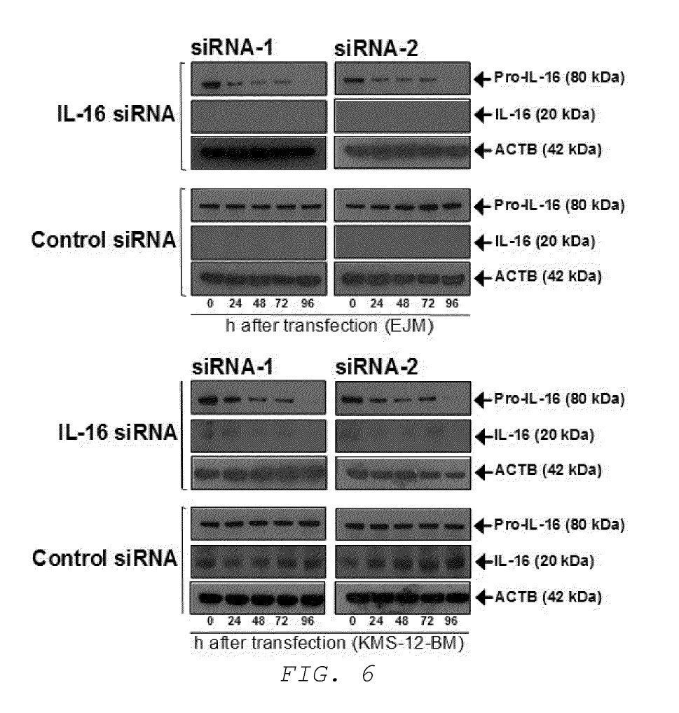

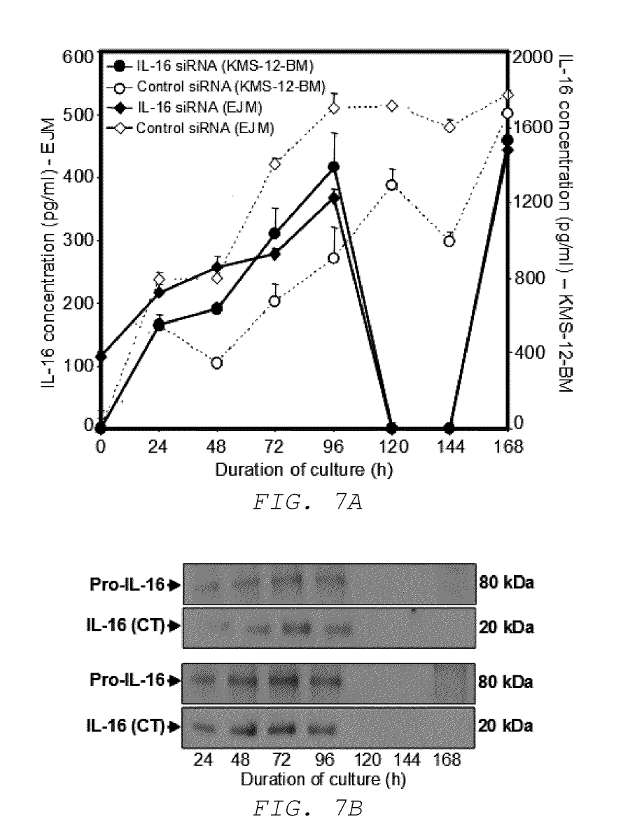

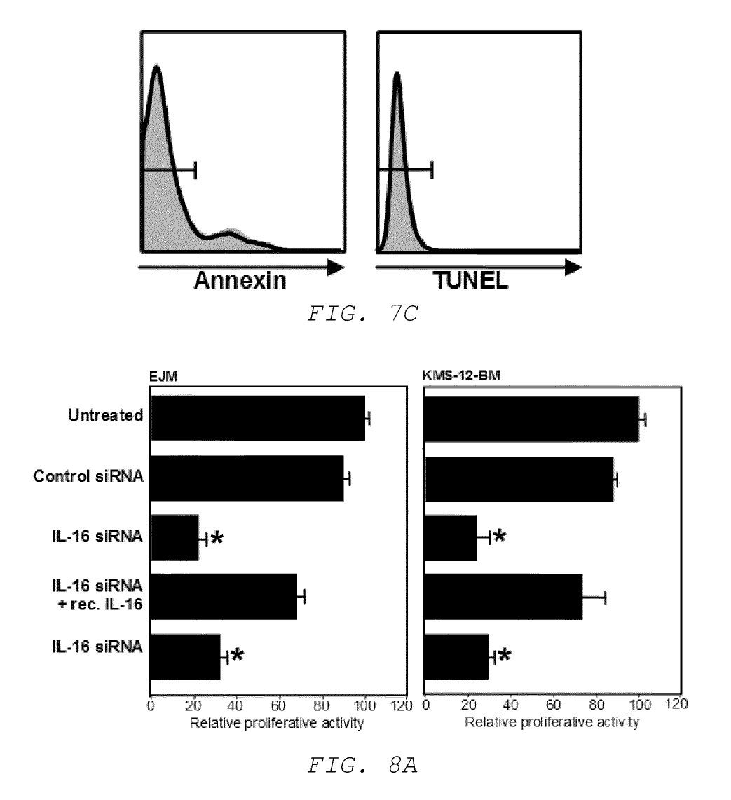

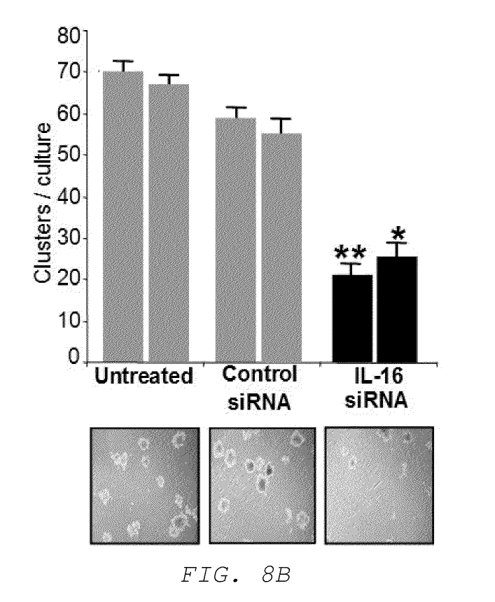

Using inhibitory RNA, the inventors for the first time demonstrated the function of IL-16 in malignancies such as multiple myeloma. They showed that silencing of IL-16 gene expression (FIG. 6A) leads to a decreased secretion of soluble IL-16 by myeloma cells (FIGS. 7A+B). As a consequence, myeloma cell proliferation significantly decreases (FIG. 8A). This phenomenon is independent from the occurrence of spontaneous apoptosis (FIG. 7C) and also reduces the growth of myeloma precursors—so-called myeloma stem cells (FIG. 8B).

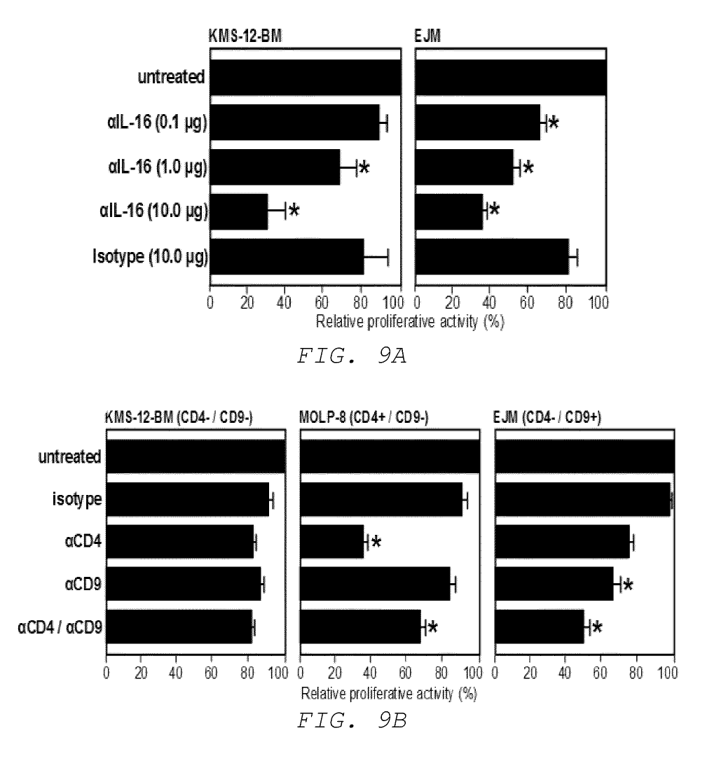

Most importantly, the inventors showed for the first time that the addition of an anti-IL-16 antibody to myeloma cell cultures led to a concentration-dependent inhibition of myeloma cell growth (FIG. 9A). An isotype antibody added at maximal concentration had no such an effect. Furthermore, the inventors demonstrated that monoclonal antibodies against the IL-16 receptors CD4 and CD9 had a comparable inhibiting effect on the proliferation of myeloma cells (FIG. 9B). Overall, the findings of the inventors suggest that antibody-mediated therapies directed against IL-16 and its receptors could have significant activity as therapies for patients with multiple myeloma.

The inventors have shown for the first time that soluble IL-16 is secreted by tumor cells from patients with hematological malignancies, such as multiple myeloma. IL-16 secretion by the malignant cells leads to increased concentrations of IL-16 at the tumor site, i.e. the bone marrow, and in the peripheral blood of the respective patient when compared to healthy controls. Analysis of IL-16 expression, i.e. by flow cytometry using an appropriate monoclonal antibody, improves the identification of the malignant cells. Downregulation of IL-16 production by gene silencing results in a growth reduction of the tumor cells and a monoclonal antibody against IL-16 or its receptors can be used to inhibit proliferation of the malignant cells. The invention opens the route for new applications for IL-16, for example as a target for monoclonal antibodies in diagnosis and therapy. Such tumor-specific antibodies can be used for diagnostic purposes or in novel and promising modes of therapy for patients with solid tumors and, in particular, patients with hematological malignancies such as multiple myeloma.

### EXAMPLES

### Cell Lines

Myeloma cell lines MOLP-8, RPMI-8226, KMS-12-BM, EJM; IM-9, U-266, NCI-H929, OPM-2, and LP-1 were obtained from the German Collection of Microorganisms and Cell Cultures (DSMZ, Braunschweig, Germany). Cell lines Brown, U-266, and SK-007 were provided by the New York branch of the Ludwig Institute for Cancer Research (LICR). Lines were maintained in RPMI 1640 with penicillin/streptomycin and 10% or 20% FCS, respectively. For evaluation of IL-16 concentrations within cell cultures, supernatants were removed 48 h (and in some cases 24 h, 72 h, 96 h, 120 h, 144 h, 168 h) after culture initiation and samples were frozen at −80° C. until final analysis. For some experiments, granulocyte-macrophage colony-stimulating factor (GM-CSF), interferon alpha (INF-•), a proliferation-inducing ligand (APRIL), tumor necrosis factor-alpha (TNF-•), insulinlike growth factor (IGF), B cell activation factor of the TNF family (BAFF), IL-1•, IL-6, IL-10, and IL-17 were added at different concentrations (10, 50, 250 ng/ml) 48 h after culture initiation and effects on IL-16 production and cellular growth were evaluated 24 h later.

### Patients and Healthy Donors

In total, 62 consecutive myeloma patients, one patient with monoclonal gammopathy of undetermined significance (MGUS), 8 healthy BM donors, and 6 healthy blood donors were studied. Tonsillar tissue was obtained from 7 adult patients undergoing tonsillectomy for chronic tonsillitis. BM samples from MM patients were obtained during routine diagnostic procedures. Whole BM samples obtained from consented healthy donors were part of BM donations for alloSCT. MM patients evidenced at least 10% BM-infiltrating myeloma cells as defined by CD138/CD38 co-expression and as confirmed by conventional cytological examination. Healthy subjects and patients, who were admitted for treatment at the University Medical Center Hamburg-Eppendorf, gave informed consent in accordance with the revised version of the Declaration of Helsinki. The study protocol had been approved by the local ethics committee (decision number OB-038/06).

### Preparation of Tissue, Blood, and BM Samples

Tonsillar tissue was manually chopped into small pieces and a cell suspension was prepared using a Medimachine (BD Biosciences, San Jose, Calif.). For preparation of plasma samples whole BM or PB was centrifuged the at 1800 rpm and supernatants were removed and frozen at −80° C. until final analysis. Mononuclear cells (MNC) were isolated f cases, CD8+ T cells were enriched from whole PBMC applying a magnetic micobead rom blood and BM samples by density gradient centrifugation and, in some -based technique (Milteny Biotec, Bergisch Gladbach, Germany)

### Human Cytokine Array and Enzyme-linked Immunosorbent Assay (ELISA)

The Human Cytokine Array is a part of the Proteome Profiler™ platform (R&D Systems Inc., Minneapolis, Minn.). This array allows for simultaneously profiling the relative levels of 36 different cytokines/chemokines in a single sample using nitrocellulose membranes on which selected capture antibodies are spotted in duplicate. In brief, supernatant of cell line EJM was harvested 48 hours after culture initiation and was diluted with standard array buffer. After addition of a cocktail of biotinylated. detection antibodies, samples were added to the nitrocellulose membranes coated with capture antibodies and were incubated overnight at 4° C. After washing, streptavidin-HRP was added and, following an additional 30-minute incubation period, each membrane was developed with a chemiluminescent detection. reagent. Membranes were exposed to an X-rav film for 10 minutes and chemiluminescence was quantified by scanning the developed X-ray film on a transmission-mode scanner. The array was repeated three times in order to guarantee reproducibility of the results.

Quantitative analysis of IL-16 concentrations in cell culture supernatants and plasma samples derived from bone marrow and peripheral blood was performed using a commercially available Quantikine kit (R&D systems) according to the manufacturer's instructions. Following development of the ELISA plates, absorbance was read at 450 nm using a spectrophotometer (Tecan, Mannedorf, Switzerland). IL-16 concentrations were interpolated from a standard curve, which was generated using the respective recombinant protein.

### Conventional and Real-time Reverse Transcription Polymerase Chain Reaction (RT-PCR)

Extraction of total RNA from tonsillar tissue, cell lines, BM, PBMC, and CD8+ T cells was performed using the RNeasy Mini Kit (Qiagen, Hilden, Germany) and transcription was done using AMV reverse transcriptase (Promega, Madison, Wis.). RNA derived from a set of 20 different healthy tissues was obtained from Ambion (Austin, Tex.). Conventional and quantitative PCR were performed as previously described (Atanackovic, Luetkens et al. 2009). Primers for the detection of IL-16 and housekeeping gene glyceraldehyde-3-phosphate dehydrogenase (GAPDH; and by conventional and/or real-time PCR are known in the state of the art (e.g., Cho et al. 2008) and were obtained from MWG Biotech (Ebersberg, Germany) and Qiagen, respectively. Results for real-time PCR experiments are given as copies of the target gene IL-16 in relation to copies of housekeeping gene GAPDH. All RT-PCR experiments were performed at least twice. To assess primer specificity, PCR products were analyzed repeatedly by sequence analysis.

### Western Blot

Whole cell protein extracts were prepared from cell lines, bone marrow MNC, and CD8+ T cells enriched from whole PBMC using PBS with 1% Igepal CA-630, 0.5% sodium-deoxycholate, and 0.1% SDS containing a cocktail of protease Inhibitors (Sigma, Steinheim, Germany). Cell culture supernatants as well as BM and PB plasma samples from myeloma patients and healthy donors were used undiluted or diluted 1:100. Western blotting was performed using 30 μg protein/lane and applying a primary antibody against IL-16, which recognizes the C-terminal mature part of the protein (Clone 70719; R&D Systems), a monoclonal antibody against ACTB (Santa Cruz Biotechnology) and a secondary HRP-labeled anti-mouse monoclonal antibody (R&D Systems). Specific binding was visualized by chemiluminescence (Amersham Biosciences). For all target proteins analyzed, appropriate blocking studies were undertaken using recombinant proteins in order to confirm specificity of the staining.

Gene Silencing Using Transfection with Stealth RNAi

Non-targeting GFP-coupled stealth RNAi, scrambled control RNAi, and validated stealth RNAi targeting IL-16 were purchased from Invitrogen (Carlsbad, Calif.). Specific down-regulation of IL-16 was achieved with two out of three of the commercially available RNAis (HSS142654 and HSS142656), respectively.

Myeloma cell lines were transfected using the cationic lipid-based reagent Lipofectamine 2000 (Invitrogen). For each condition, 3×10⁵ cells were washed and resuspended in 100 μl Optimem I medium (Gibco, Karlsruhe, Germany). 50 pmol of stealth RNAi with or without 1 μl Fluorescent Control (Invitrogen) was added to the cells and incubated for 10 minutes at room temperature. Lipofectamine 2000 was gently mixed before being used and was diluted 1:40 in Optimem I medium without serum followed by an incubation at room temperature for 10 minutes. 100 μl of the Lipofectamine 2000 dilution was then added to the cells and incubated at room temperature for 20 minutes. Next, cell suspensions were transferred to a 24-well plate (Greiner Bio-One, Frickenhausen, Germany) and incubated at 37° for 4 hours. Afterwards, 1.5 ml complete medium was added and cells were cultured at 37° C. for another 72 hours. Cells were stained with nuclear and dead cell stain (RNAi Basic Control Kit-Human; Invitrogen) and transfection efficiency was evaluated using 40× bright-field microscopy. Images were obtained using a digital camera (Canon, Krefeld, Germany) and Adobe Photoshop CS3 imaging software (Adobe Systems Inc., San Jose, Calif., USA). Transfection efficiency was generally 70-80% and cell death less than 5% at 24 h post transfection as determined by fluorescent microscopy.

### Flow Cytometry

For the analysis of cytoplasmatic IL-16 protein expression, myeloma cell lines or bone marrow MNC were first stained using a CD138-FITC monoclonal antibody (clone B-A38, BD Biosciences). Next, cells were fixed using FACS Lysing Solution (BD Biosciences) and were permeabilized using Permeabilizing Solution (BD Biosciences). Cytoplasmatic staining was performed applying a PE-conjugated anti-IL-16 antibody (clone 14.1, BD Biosciences) or an appropriate isotype control. Samples were analyzed using a FACSCalibur cytometer (BD Biosciences) and FlowJo software (Tree Star, Ashland, Oreg.).

Two different flow cytometric assays for the determination of levels of apoptosis were used: The TUNEL (terminal deoxynucleotidyl transferase dUTP nick end labeling) assay was done according to the manufacturer's recommendations (Millipore, Billerica, Mass). Cells were fixed for in 1% paraformaldehyde, were permeabilized and, following two washes, were incubated in staining solution containing 8 μl Fluorescein-dUTP at 37° C. for one hour. Cells were then washed and incubated with PI/RNAse A solution for 30 minutes at room temperature. Analysis by flow cytometry was performed within the next three hours. For Annexin staining, 1×10⁶ cells were incubated with Annexin V FITC in binding buffer. Before flow cytometric analysis the reaction was stopped with 900 μl Annexin binding buffer.

### Analysis of Cell Proliferation

The proliferative rate of myeloma cells was usually assessed 72 h after transfection with IL-16-specific RNAi. In some experiments, anti-IL-16 (clone 70719, R&D Systems), anti-CD4 (clone 10C12, Abcam, Cambridge, Mass.) and/or anti-CD9 (clone MEM61, Abcam) blocking antibodies were or were added at a concentration of 1 μg/ml before the initiation of the proliferation assay. Rescue experiments were performed adding recombinant IL-16 protein (R&D Systems) to the cell culture at a concentration of 5 μg/ml. In the Biotrak™ ELISA proliferation read-out assay (Amersham Biosciences) myeloma cells were pulsed with 10 μM Bromodeoxyuridine (BrdU) for the last 18 hours of culture. Following fixation, peroxidase-labelled anti-BrdU, which binds to the BrdU incorporated into newly synthesized cellular DNA, was added. Resulting immune complexes were detected by a substrate reaction, and absorbance was read at 450 nm using a microtiter plate spectrophotometer (Tecan).

### Colony Formation Assay

Myeloma cell lines EJM and KMS-12-BM were plated at 1000 cells/ml of methylcellulose medium (StemCell Technologies, Cologne, Germany) and 1 ml/well in a 6-well culture dish (Nunc, Langensebold, Germany). Plates were incubated at 37° C. and colonies consisting of >40 cells were counted at 10 days after culture initiation.

### Statistical Analysis

Statistical analyses were performed using SPSS software. The Mann-Whitney U test was used to calculate differences between different experimental conditions. Differences were regarded significant if p<0.05.

### REFERENCES

1) DE 196 49233
2) DE 19617203
3) WO 2006060598
4) Alonsi et al., 2007, Progress in Chemokine Research, W. P. Linkes Ed.:157-68
5) Bellomo et al., 2007, Leukemia & Lymphoma 48(6):1225-7
6) Alexandrakis et al., 2004, American Journal of Hematology 75(2):101-6
7) Koike et al., 2002, Leukemia Research 26(8):705-11
8) Magrangeas et al., 2003, Blood 101(12):4998-5006
9) Cao et al., 2009, ASH Annual meeting, New Orleans, December 2009
10) Baier et al., 1997, Proceedings of the National Academy of Sciences USA 94:5273-5277
11) Cho et al., 2008, Experimental and Molecular Medicine 40(2):237-245
12) Atanackovic, Luetkens et al., 2009, Clinical Cancer Research 15(4):1343-1352

1. A method of treating a hematological malignancy or a solid tumor or of preventing progression from a premalignancy to a malignancy, comprising administering to a subject an effective amount of an agent capable of specifically targeting IL-16.

2. The method of claim 1, wherein the hematological malignancy is multiple myeloma (MM), indolent myeloma, smoldering myeloma, acute myeloid leukemia (AML), chronic myeloid leukemia (CML), acute lymphatic leukemia (ALL), chronic lymphatic leukemia (CLL), cutaneous T-cell leukemia (e.g., Sezary syndrome), hairy cell leukemia, Hodgkin's disease, Non-Hodgkin lymphoma, myelodysplastic syndrome (MDS) or myeloproliferative disease.

3. The method of claim 1, wherein the premalignancy is monoclonal gammopathy of undetermined significance (MGUS).

4. The method of claim 1, wherein the solid tumor is colon carcinoma, breast carcinoma, lung carcinoma (small-cell lung cancer and non-small cell lung cancer), head and neck cancer, glioblastoma multiforme, sarcoma, cervical cancer, endometrial cancer, cancer of the pancreas, thyroid, stomach, bladder, skin, breast, prostate, ovary, kidney, or liver.

5. The method of claim 1, wherein the agent is an anti-IL-16 antibody, a soluble 1L-16 receptor or an antibody directed against an IL-16 receptor.

6. The method of claim 5, wherein the agent is an anti-IL-16 antibody or a soluble IL-16 receptor and it is coupled to an active compound selected from the group comprising a cytotoxic compound.

7. The method of claim 1, wherein the agent targeting IL-16 is an IL-16 receptor antagonist or an IL-16 TRAP.

8. The method of claim 1, wherein the agent targeting IL-16 inhibits IL-16 expression on the RNA and/or protein level.

9. The method of claim 8, wherein the agent is inhibitory RNA or antisense RNA.

10-19. (canceled)
